(19) United States
(12) Patent Application Publication
(10) Pub. No.: US 2026/0020796 A1
(43) Pub. Date: Jan. 22, 2026

(54) SENSOR INSERTION APPARATUS FOR VARIABLE ANGLE SENSOR INSERTION

(71) Applicant: Konamite Limited, Dublin (IE)

(72) Inventors: Mihailo V. Rebec, Portland, OR (US); Robert Bruce, Portland, OR (US)

(21) Appl. No.: 19/263,938

(22) Filed: Jul. 9, 2025

Related U.S. Application Data
(60) Provisional application No. 63/673,220, filed on Jul. 19, 2024.

(51) Int. Cl. A61B 5/1486; A61B 5/145

(52) U.S. Cl. CPC A61B 5/14865; A61B 5/14532; A61B 2560/063

(57) ABSTRACT

Embodiments provide for insertion devices for flexible analyte sensors that enable a user to select from a wide variety of different angles of sensor insertion. In an example, an insertion device comprises a guidance structure for supporting the flexible analyte sensor during insertion into skin, and an insertion angle adjustment component configured to manipulate the guidance structure that in turn adjusts the angle of flexible analyte sensor delivery to skin. In this way, insertion angle may readily be changed by a user or technician, depending on the application.

### CLAIM OF PRIORITY

This application claims priority under 35 USC § 119(e) to U.S. Provisional Patent application Ser. No. 63/673,220, filed on Jul. 19, 2024, the entire contents of which are hereby incorporated by reference.

### TECHNICAL FIELD

Embodiments herein relate to analyte sensors, and more specifically, to devices for the insertion of analyte sensors at user-defined variable angles.

### BACKGROUND

Implantable or semi-implantable/percutaneous analyte sensors offer a convenient and accurate alternative to analyte testing methods such as fingerstick analyte meters (e.g., blood glucose meters). Indwelling sensors also offer an additional advantage in that analyte concentrations in an individual may be tracked continuously over a period of time without requiring the individual to draw a blood sample for each measurement. Where close attention to blood analyte concentrations is correlated with better outcomes, an indwelling analyte sensor may be superior to other monitoring options. In diabetes, for example, blood glucose levels that are either higher or lower than a target level may result in serious medical complications. Continuously monitoring blood glucose levels using an implanted sensor and an external electronic measuring device may improve the user's ability to control blood glucose levels, thus reducing the incidence and severity of such complications.

Flexible indwelling sensors may be more comfortable for the user than rigid or semi-rigid sensors, and less likely to experience failure due to mechanical stress induced by the user's movements. However, the insertion of flexible sensors requires an initial piercing of the skin due to the tendency of flexible sensors to bend. Therefore, insertion of flexible sensors is often accomplished by inserting the sensor through a rigid hollow mechanism such as a needle, a cannula or a trocar used to create a channel through which the sensor could pass.

The use of such piercing mechanisms for sensor insertion may cause physical and emotional discomfort among users, discouraging the use of indwelling sensors for continuous blood analyte monitoring. Piercing mechanisms that accommodate a sensor may be large and painful to insert through skin. In addition to the physical discomfort associated with piercing mechanisms, an individual in need of consistent blood analyte monitoring may be reluctant to use such a mechanism without assistance.

Analyte sensor flexibility reduces user discomfort in long-term sensor use. But as flexibility increases, the capacity of the sensor to directly penetrate unbroken skin diminishes. While flexible indwelling analyte sensors may improve a user's ability to monitor blood analyte levels for optimal medical outcomes, the available insertion methods may be unacceptable to some who are in need of monitoring.

### BRIEF DESCRIPTION OF THE DRAWINGS

Embodiments will be readily understood by the following detailed description in conjunction with the accompanying drawings and the appended claims. Embodiments are illustrated by way of example and not by way of limitation in the figures of the accompanying drawings.

FIG. 1 illustrates an example analyte sensor system, in accordance with various embodiments;

FIG. 2 illustrates an example flexible analyte sensor insertion device, for use to practice various embodiments;

FIGS. 3A-3B depict an example embodiment of how an angle of insertion for flexible analyte sensors of the present disclosure may be changed;

FIGS. 4A-4E depict another example embodiment of how the angle of insertion for flexible analyte sensors of the present disclosure may be changed;

FIGS. 5A-5D depict yet another example embodiment of how the angle of insertion for flexible analyte sensors of the present disclosure may be changed;

FIGS. 6A-6D illustratively depict how a tensioning structure may be combined with embodiments of the present disclosure that enable variable flexible analyte insertion angles;

FIG. 7 depicts a perspective view of a tensioning structure to illustrate how the tensioning structure avoids obstructing insertion of flexible analyte sensors of the present disclosure;

FIGS. 8A-8B depict an example embodiment of a guide structure of the flexible analyte sensor insertion devices of the present disclosure that includes electrical contacts which selectively electrically couple to electrical contacts associated with a flexible analyte sensor;

FIGS. 9A-9B depict an example embodiment of a flexible analyte sensor insertion device that relies on an elastic nature of skin to cause insertion of a flexible analyte sensor into skin;

FIGS. 10A-10B depict an example where a skin tensioning structure includes two components, a first skin tensioning structure and a second skin tensioning structure, to impart skin tensioning along differing dimensions with respect to skin; and

FIGS. 11A-11C depict example illustrations of an embodiment of a second skin tensioning structure (FIGS. 11A-11B), and a positional relationship (FIG. 11C) between the first skin tensioning structure and the second skin tensioning structure.

### DETAILED DESCRIPTION OF DISCLOSED EMBODIMENTS

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense.

Various operations may be described as multiple discrete operations in turn, in a manner that may be helpful in understanding embodiments; however, the order of description should not be construed to imply that these operations are order-dependent.

The description may use perspective-based descriptions such as up/down, back/front, and top/bottom. Such descriptions are merely used to facilitate the discussion and are not intended to restrict the application of disclosed embodiments.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A) B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous, and are generally intended as “open” terms (e.g., the term “including” should be interpreted as “including but not limited to,” the term “having” should be interpreted as “having at least,” the term “includes” should be interpreted as “includes but is not limited to,” etc.).

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity.

Discussed herein, the term “actuator” refers to any of various electric, hydraulic, magnetic, pneumatic, mechanical, or other means by which something is moved or controlled. The term “solenoid actuator” refers to a variety of electromechanical devices that convert electrical energy into linear or rotational motion. The term “trigger” indicates any of various electric, hydraulic, magnetic, pneumatic, mechanical, or other means of initiating a process or reaction.

For the purposes of describing embodiments herein and in the claims that follow, the term “associated with” indicates that an object, element, or feature is coupled to, connected to, or in proximity to and in communication with another object, element, or feature.

For the purposes of describing embodiments herein and in the claims that follow, the term “guide structure” means a device that at least partially axially surrounds the flexible analyte sensors of the present disclosure, whether at an end or along the sensor. The flexible analyte sensors of the present disclosure may be adapted to fit inside the guidance structure such that the guide structure at least partially occupies at least some part of the space between the sensor and the guidance structure either during insertion, before insertion, and/or after insertion. A guide structure may either provide axial and/or radial support, assist a sensor in moving through the guidance structure, or both. Exemplary guide structures may include but are not limited to a tubing that is open on either end (e.g., flexible, rigid, or some combination), a wall (e.g., a walling that is rigid or at least somewhat flexible/malleable), or any other structure capable of providing support to a flexible analyte structure as it is inserted into animal skin. For example, a guide structure may be made of many different materials and shaped in various geometries. In embodiments, the geometry is variable to accommodate variable flexible analyte insertion angles.

For the purposes of describing embodiments herein and in the claims that follow, the term “electrical network” means electronic circuitry and components in any desired structural relationship adapted to, in part, receive an electrical signal from an associated sensor and, optionally, to transmit a further signal, for example to an external electronic monitoring unit that is responsive to the sensor signal. The circuitry and other components include one or more of a printed circuit board, a tethered or wired system, etc. Signal transmission may occur over the air with electromagnetic waves, such as RF communication, or data may be read using inductive coupling. In other embodiments, transmission may be over a wire or via another direct connection.

Discussed herein, flexible analyte sensors may comprise sensors such as those described in U.S. Pat. No. 5,965,380 to Heller et al. and U.S. Pat. No. 5,165,407 to Ward et al., which are hereby incorporated by reference in their entirety. For example, the flexible analyte sensors may comprise sensors used in continuous glucose monitoring systems, where an enzyme layer (e.g., glucose oxidase) catalyzes oxidation of glucose that in turn ultimately results in analyte sensor current. While glucose sensors are disclosed as a representative example, the devices herein disclosed equally apply to other analyte sensors, including but not limited to other enzyme-based sensors, DNA-based sensors, thermal and/or piezoelectric biosensors, and the like, capable of measuring any number of different analytes.

Thus, embodiments herein provide for an insertion device for delivering an analyte sensor into skin. The insertion device may include a guidance structure that at least partially supports the analyte sensor during its insertion into skin. The insertion device may include an insertion angle adjustment component configured to adjust an angle at which the analyte sensor is driven into skin.

In an example, the analyte sensor may comprise a flexible analyte sensor, with a buckling force of less than 0.25 Newtons. In some examples the insertion angle adjustment component may adjust the angle at which the analyte sensor is driven into the skin by changing a shape of the guidance structure. In other examples, the insertion angle adjustment component may adjust the angle at which the analyte sensor is driven into skin by changing a positioning of the guidance structure without altering its shape. An insertion activation device may be configured to provide a high speed motive force to the analyte sensor, to drive the analyte sensor into skin.

In some examples, the insertion device may comprise an adjustable skin tensioning structure configured to tension skin prior to delivering the analyte sensor into skin. The insertion angle adjustment component may coordinate adjustment of the adjustable tensioning structure as a function of the angle at which the analyte sensor is driven into skin. For example, tensioning skin via the adjustable tensioning structure may include depressing skin to a variable extent, where the skin is depressed to a greater extent as the angle at which the analyte sensor is driven into skin is reduced (e.g., 10° with respect to an external plane of skin), and wherein the skin is depressed to a lesser extent as the angle at which the analyte sensor is driven into skin is increased (e.g., 90° with respect to the external plane of skin). Accordingly, in examples, the angle at which the analyte sensor is driven into skin may be selectable from 10° to 90° with respect to the external plane of skin.

In another example embodiment, an insertion device for delivering a flexible analyte sensor into skin comprises an insertion angle adjustment component configured to adjust an angle at which the flexible analyte sensor is delivered into skin, and a guidance structure operably coupled to the insertion angle adjustment component. In such an example, the angle at which the flexible analyte sensor is delivered into skin may be adjusted via the insertion angle adjustment component by changing a shape of the guidance structure. Changing the shape of the guidance structure may adjust the angle at which the flexible analyte sensor is delivered into skin from 10° to 90° with respect to an external plane of skin. In some examples, the angle may be adjusted in a continuously variable manner. In other examples, the angle may be adjusted in a step-wise manner. The flexible analyte sensor may be delivered to skin along a linear path or a curved path depending on the shape of the guidance structure.

In examples of the insertion device, the guidance structure may be configured to receive a high speed motive force from an insertion activation device. The high speed motive force may comprise a force that causes movement of the flexible analyte sensor that ultimately delivers the flexible analyte sensor into skin. In examples, the insertion activation device remains fixed in terms of position regardless of the angle at which the flexible analyte sensor is delivered into skin. In such an example, a direction at which the high speed motive force is produced via the insertion activation device relative to an external plane of skin may not change, regardless of the angle at which the flexible analyte sensor is delivered into skin. The high speed motive force may be provided mechanically, pneumatically, magnetically, or hydraulically.

In some examples, the insertion device may further comprise an adjustable skin tensioning structure configured to depress skin to a variable extent as a function of the angle at which the flexible analyte sensor is delivered into skin. The insertion angle adjustment component may simultaneously coordinate movement of the skin tensioning structure with changing of the shape of the guidance structure. The adjustable skin tensioning structure may depress skin to the variable extent as the function of the angle at which the flexible analyte sensor is delivered into skin in order to ensure the flexible analyte sensor perpendicularly penetrates skin regardless of the angle at which the flexible analyte sensor is delivered into skin. In examples, the guidance structure may include an exit port through which the flexible analyte sensor travels in order to be delivered into skin. The skin tensioning structure may be associated with the exit port of the guidance structure, and may include a dedicated slot through which the flexible analyte sensor travels through during delivery into skin.

In some examples, the guidance structure includes one or more integrated electrical contacts capable of electrically coupling to a portion of the flexible analyte sensor that remains external to skin. The integrated electrical contacts may further electrically couple the sensor to other componentry, for example a control unit that includes a transmitter for transmitting sensor readings to an external device. In such an example, the electrical coupling may occur as a result of (e.g., during the process of) delivering the flexible analyte sensor into skin. In other examples, the guidance structure may include one or more openings that enable electrical connections between the flexible analyte sensor and, for example, the control unit/transmitter, to be established either before insertion of the flexible analyte sensor into skin, or after the flexible analyte sensor has been inserted into skin.

In yet another example embodiment, an insertion device for delivering a flexible analyte sensor into skin comprises a guidance structure configured to at least partially surround the flexible analyte sensor and a housing that at least partially surrounds a portion of the guidance structure. The insertion device may further include a first spring that couples the housing to the guidance structure, a pin configured to lock the housing to the guidance structure to prevent relative movement of the guidance structure and the housing, and a second spring associated with the pin. The insertion device may further comprise a notch that maintains the flexible analyte sensor at a first position relative to the guidance structure under conditions where the housing is locked via the pin to the guidance structure.

In examples of such an insertion device, compression of the second spring may unlock the housing and the guidance structure, such that guidance structure is able to move with respect to the housing. The movement may occur at a speed that is a function of one or more parameters of the first spring. Such parameters may include spring outside diameter, spring inside diameter, non-compressed (unloaded) spring length, fully compressed spring length (e.g., minimum length), spring pitch, compression spring rate (e.g., determined by an amount of force required to constrict the spring by a predetermined length), spring material composition, number of coils, etc.

In examples, movement of the guidance structure with respect to the housing may overcome a holding force of the notch. This in turn may position the flexible analyte sensor at a second position with respect to the guidance structure. The guidance structure may include an exit port, wherein no part of the flexible analyte sensor crosses a plane of the exit port when the flexible analyte sensor is in the first position. Alternatively, at least a portion of the flexible analyte sensor may cross the plane of the exit port to extend past the guidance structure when the flexible analyte sensor is in the second position.

Other embodiments will be made apparent from the following detailed disclosure.

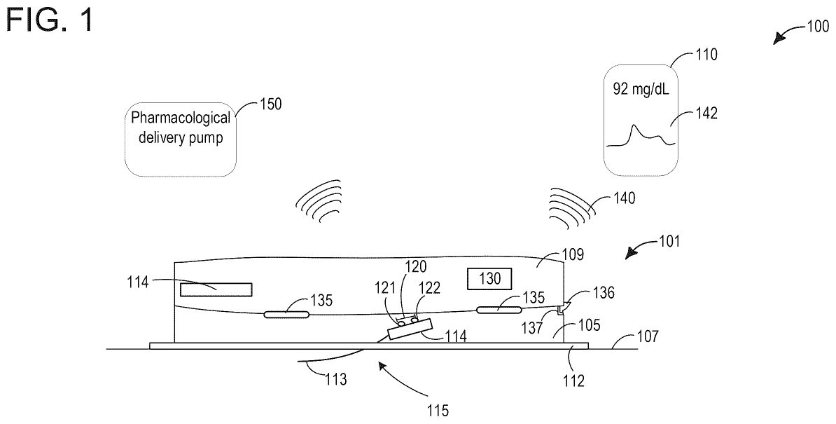

Turning to FIG. 1, depicted is an example analyte sensor system 100 relevant to the present disclosure. Analyte sensor system 100 includes sensing device 101. Sensing device 101 is comprised of a sensor base 105 which at least partially houses flexible analyte sensor 115. “Flexibility” as discussed herein with regard to analyte sensors refers to the amount of deflection of an elastic body for a given applied force. When implanted into skin (e.g., subcutaneously implanted), analyte sensors of the present disclosure may flex repeatedly (e.g., hundreds of times or more) as a result of normal movement of the subject, without fracture, for a predetermined period of time (e.g., 3-14 days or more). Flexible analyte sensors of the present disclosure may be inserted into skin without the assistance of a trocar, cannula, lancet, needle, or other similar device, thereby reducing pain, discomfort, possibility of infection, etc. In examples a buckling force of the analyte sensors 115 of the present disclosure may be less than 0.25 Newtons, or less than 0.1 Newtons, or less than 0.05 Newtons.

Sensor base 105 may be secured to skin 107 of a subject via adhesive patch 112. In some examples, adhesive patch 112 may comprise a skin tensioner functionality, which may facilitate sensor insertion in some examples. Specifically, adhesive patch 112 may be less elastic than skin 107. When adhered to skin 107 around a sensor insertion site, skin stretching may be reduced which may in turn limit an amount of indentation of skin 107 upon sensor insertion. This may be advantageous in facilitating sensor insertion without undesirable sensor deformation (e.g., bending, buckling, etc.).

Flexible analyte sensor 115 may comprise a biosensor (e.g., electrochemical glucose sensor) that has been fabricated onto a length of thin, flexible wire. While not explicitly illustrated at FIG. 1, it may be understood that a reference electrode (e.g., Ag/AgCl wire) and a sensing electrode are incorporated into analyte sensor 115. The reference electrode and the sensing electrode may be incorporated into a small diameter end 113 of analyte sensor 115. Small diameter end 113 may be inserted through skin 107 of a subject. Small diameter end 113 may be as small as 10 μm in diameter in some examples. In some examples, small diameter end 113 may be between 10-25 μm in diameter. In still other examples, small diameter end 113 may be greater than 25 μm in diameter, for example between 25-50 μm, or 50-75 μm, or 75-100 μm in diameter.

Larger diameter end 114 of analyte sensor 115 is of a larger diameter than small diameter end 113 as a result of the addition of a sleeve of steel tubing. The steel tubing may increase rigidity and facilitate electrical connections. Larger diameter end 114 may be 500 μm or less in diameter, for example, such as between 250-500 μm in diameter. In some examples, larger diameter end 114 may be less than 250 μm in diameter, for example between 100-225 μm in diameter. As depicted at FIG. 1, larger diameter end 114 remains outside of the body of the subject (external to skin 107). In-skin sensor length as herein disclosed may be between 1 mm and 10-12 mm. A depth to which analyte sensors of the present disclosure penetrate skin may be variable as a function of an angle at which the sensor is inserted into the skin of a subject, and length of small diameter end 113 (and overall length of sensor 115). Discussed herein, small diameter end 113 (e.g., the inserted portion) is referred to as a distal end, and larger diameter end 114 is referred to as a proximal end of analyte sensor 115.

Larger diameter end 114 includes a control unit coupling portion 120 that couples analyte sensor 115 to control unit 109. Specifically, control unit coupling portion may include electrical contacts, such as sensing electrode contact 121 and reference electrode contact 122. Sensing electrode contact 121 may be connected to the sensing electrode and reference electrode contact 122 may be connected to the reference electrode. The electrical contacts may be in any configuration that allows for contact within the control unit, for example flush or annular rings. In some examples, a conductive polymer may be used to electrically couple flexible analyte sensor 115 to appropriate electrical circuitry associated with the control unit 109. Examples of conductive polymer relevant to the present disclosure can include but are not limited to polypyrrole [PPy], polyaniline, poly(3,4-ethylenedioxythiophene) (PEDOT), polythiophene (PT), etc. Advantages to using a conductive polymer may include but are not limited to reducing weight of the sensing device 101, reduction in energy usage, corrosion resistance, reduction in size of the sensing device, etc. Furthermore, conductive polymers may be flexible, which in some examples may be advantageous in terms of accommodating the variable sensor insertion angles enabled by devices and methods of the present disclosure. Additionally, using a conductive polymer may reduce the noise present in sensor readings by providing more consistent contact between the flexible analyte sensor 115 and the electrical circuitry of the control unit 109.

Control unit 109 may be removably coupled to sensor base 105. For example, sensing device 101 may be assembled by slidingly engaging control unit 109 with grooves 135 on sensor base 105. In some examples, locking latch 136 secures to locking edge 137 to further secure control unit 109 to sensor base 105. In an embodiment, control unit 109 comprises a transmitter 130. In some embodiments, control unit 109 and transmitter 130 may comprise a reusable portion of sensing device 101, whereas sensor base 105 may comprise a disposable portion. In some examples, battery 114 may be included as part of control unit 109, however in other examples batter 114 may be included in sensor base 105. Control unit 109 may further comprise other electronic circuitry (e.g., processing components, memory, non-transitory computer-readable medium, etc.).

Transmitter 130 may contain circuitry associated with an electrical network adapted to receive an electrical signal from flexible analyte sensor 115, and to transmit a further signal to electric monitoring unit 110 that is responsive to the sensor signal. In embodiments, such an electrical network may comprise a variety of components in any desired structural relationship. As an example, signal transmission between transmitter 130 and electric monitoring unit 110 may occur via wireless network 140 (e.g., signal transmission via electromagnetic waves, such as radiofrequency communication). In other additional or alternative examples, transmission may be via a wire or other direct connection. Electric monitoring unit 110 may include a display 142, which a user can view to retrieve information pertaining to analyte sensor 115 output. Analyte sensor output may be in the form of numbers (e.g., analyte concentration in mg/dL), graphs, etc. Electric monitoring unit 110 may be a device including but not limited to a smartphone, laptop, computer, or other computing device capable of performing the above-mentioned operations.

In some examples, analyte sensor system 100 may include a pharmacological delivery pump 150. As a representative example, where analyte sensor 115 is a glucose sensor, pharmacological delivery pump 150 may be an insulin delivery pump. Sensing device 101 may be electrically coupled to pharmacological delivery pump via wired or wireless communication, similar to that discussed above with regard to electric monitoring until 110. In some examples, pharmacological delivery pump 150 may be under direct control of sensing device 115, however in other examples at least some operations may be controlled or controllable (e.g., user-controlled) via communication between electric monitoring unit 110 and pharmacological delivery pump 150.

Thus, FIG. 1 and corresponding description pertains to an analyte sensor system 100 in which analyte sensor 115 is implanted in skin 107 of a subject. It is herein recognized that it may be advantageous to impart on such a system a functionality that enables an ability to select an angle at which analyte sensor 115 is inserted into skin 107 of a subject. For example, there may be circumstances where a user may desire to implant analyte sensor 115 at an angle of 10° with respect to the external plane of skin 107, or in other words, nearly parallel to the plane of skin 107. This may enable use of analyte sensors of the present disclosure on sites where little depth penetration is desirable, for example the leg. Specifically, the leg may not have much adipose tissue, but may have a substantial amount of muscle tissue. Thus, shallow insertion may be more desirable than deeper sensor insertion in the leg, to avoid the sensor entering into muscle.

Yet another advantage that may be realized by enabling variable angle insertion of analyte sensors of the present disclosure is an improved ability to avoid undesirable sensor placement in very young children that do not have much adipose tissue. In such subjects, a shallower angle of insertion may be desirable, to avoid entering muscle tissue in similar fashion as that discussed above.

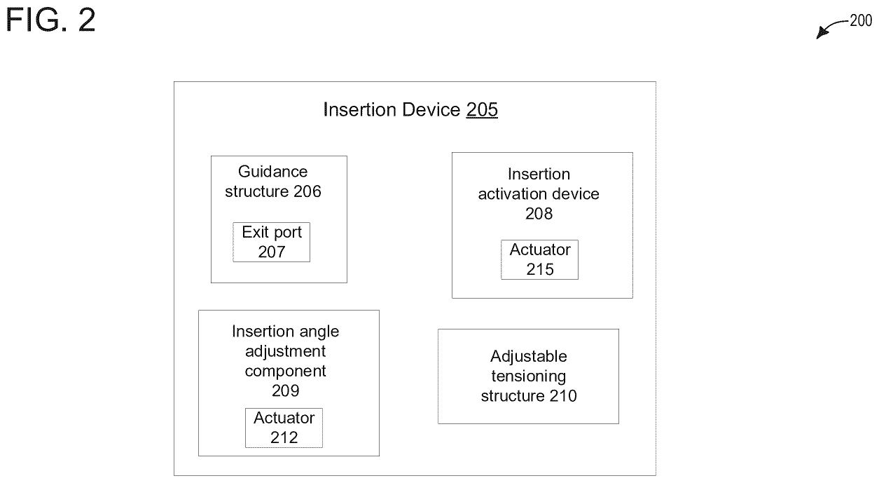

Accordingly, disclosed herein are insertion devices that enable a user to control an angle of insertion of analyte sensors of the present disclosure. Turning to FIG. 2, depicted is a block diagram of insertion device 205. Insertion device 205 is depicted as including a guidance structure 206 with an exit port 207, an insertion activation device 208, an insertion angle adjustment component 209, and an adjustable tensioning structure 210.

Guidance structure 206 may provide axial and/or radial support to flexible analyte sensors, such as flexible analyte sensor 115 depicted at FIG. 1. Insertion activation device 208 may comprise a mechanism by which a high speed motive force can be applied to flexible analyte sensor 115. An insertion activation device actuator 215 may comprise an actuator that triggers the mechanism of insertion activation device 208, to apply the high speed motive force to flexible analyte sensor 115. Upon application of the high speed motive force, the flexible analyte sensor may move at least partially through guidance structure 206, and at least partially through exit port 207, resulting in insertion of the small diameter end (e.g., small diameter end 113) into skin of a subject. It may be understood that in an example where a sensor base (e.g., sensor base 105 of FIG. 1) is included, exit port 207 may align with another exit port (not shown) on the sensor base, to enable the flexible analyte sensor to pass through the sensor base and become embedded in skin.

In some examples, guide structure 206 may comprise a tubular structure, for example a cylindrical tubing (although other tubing shapes such as square, triangular, etc., are within the scope of this disclosure). In a case where guide structure surrounds flexible analyte sensor 115, it may be understood that cross-sectional dimensions of the guide structure may be just slightly greater than (e.g., less than 10 mm difference, or less than 5 mm difference, or less than 1 mm difference) larger diameter end 114 of flexible analyte sensor 115. In this way, flexible analyte sensor may be supported and guided via the guide structure but still may enable movement of the flexible analyte sensor through the guide structure.

In some examples, the high speed motive force is such that a velocity of the flexible analyte sensor at a time at which the sensor is inserted into skin is between 1-20 meters per second. For example, the velocity may be 2 meters per second, 4 meters per second, 6 meters per second, 8 meters per second, 10 meters per second, or greater than 10 meters per second but less than 20 meters per second. In an embodiment, high speed motive force may be between 10 and 60 Newtons, for example 20 Newtons, or 30 Newtons, or 40 Newtons, etc. In some embodiments, the velocity and/or high speed motive force may be variable as a function of insertion angle of the flexible analyte sensor. In other examples, the velocity and/or high speed motive force may be fixed over a range of variable insertion angles. There may be a variety of manners in which the high speed motive force is applied to the flexible analyte sensors of the present disclosure via the insertion activation device 208. Examples include but are not limited to using energy stored in a curved sensor, a rotary solenoid, a linear solenoid, a CO₂ cartridge, an air pump and piston, mechanical spring, etc. Discussed herein, a high speed motive force refers to a force sufficient to drive a flexible analyte sensor of the present disclosure into skin, including the relatively impenetrable outer layer (stratum corneum), as well as the inner layers that are more easily penetrated, without substantial bending, deflection or buckling of the sensor. More specifically, the term “high speed motive force” encompasses any amount of motive force necessary to be applied to a thin, flexible medical analyte sensor of the present disclosure such that the sum of all forces acting on the analyte sensor as the motive force is applied is sufficient to drive it into animal skin.

According to an embodiment, insertion activation device 208 may comprise a mechanism that may be cocked to load energy into a motive force member, such as a spring, which may pull a hammer and/or plunger into a pre-insertion position. A trigger element (e.g., insertion activation device actuator 215) of the insertion activation device 208 may then be actuated, causing release of the hammer and/or plunger and the loaded energy. This may cause the hammer and/or plunger to strike or push the flexible analyte sensor assembly positioned within guide structure 206, driving the flexible analyte sensor assembly along the guide structure with sufficient force to drive the flexible analyte sensor assembly into/through intact skin without the use of a trocar or other such element that pierces the skin. In other words, only the sensing portion (e.g., smaller diameter end 113) of the flexible analyte sensor is inserted into the skin.

According to an embodiment, guidance structure 206 may be configured such that an unsupported length of the flexible analyte sensor is less than a buckling length of the sensor. The buckling length of the sensor is determined by a formula Pcr=π²*k/(3*L²), wherein Pcr is a value of the high speed motive force applied to the sensor, k is a stiffness of the sensor, and L is the unsupported length of the sensor.

In some examples, insertion device 205 may be contained within sensor base 105, and in some examples may comprise control unit 109 (and transmitter 130). In one such example where insertion device 205 is contained within sensor base 105, the flexible analyte sensor may be inserted into skin by placing the bottom of sensor base 105 on the surface of the skin, and then pressing down on control unit 109. Pressing down (e.g., in a direction towards the skin) on control unit 109 may trigger actuation of the high speed motive force that drives the flexible analyte sensor into the skin. However, in other examples, insertion device 205 may not be contained within sensor base, and may be a separate component that can be operably coupled to sensor base 105, and which can be used to trigger the high speed motive force that in turn drives the flexible analyte sensor into the skin. In some examples where insertion device 205 is not contained within sensor base 105, insertion device may be disposable. In other examples, such an insertion device 205 may be reusable.

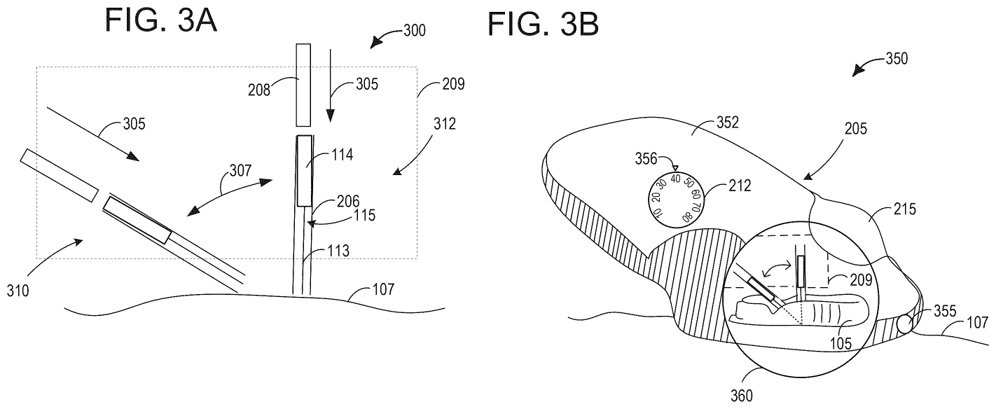

In embodiments, insertion device 205 includes insertion angle adjustment component 209. Insertion angle adjustment component 209 may enable an angle at which the flexible analyte sensor is inserted into skin to be adjusted/selected by a user. For example, insertion angle may be adjustable from about 10° with respect to a plane of skin (e.g., nearly parallel to the skin), to 90° with respect to the plane of skin, where the plane lies parallel to an external surface of the skin. In some examples, insertion angle adjustment component 209 may be configured to adjust an orientation of guide structure 207, such that the guide structure 207 moves from a first position with respect to, for example, sensor base 105 and/or a housing of insertion device 205, to a second position, without altering an overall shape of the guide structure. In such an example, a positioning of flexible analyte sensor 115 may move in coordinated fashion with guide structure 207. For example, flexible analyte sensor 115 may be positioned wholly or at least partially within guide structure 207, and hence, adjustment of guide structure 207 may cause flexible analyte sensor 115 to be repositioned. Such an example is discussed below with regard to FIGS. 3A-3B.

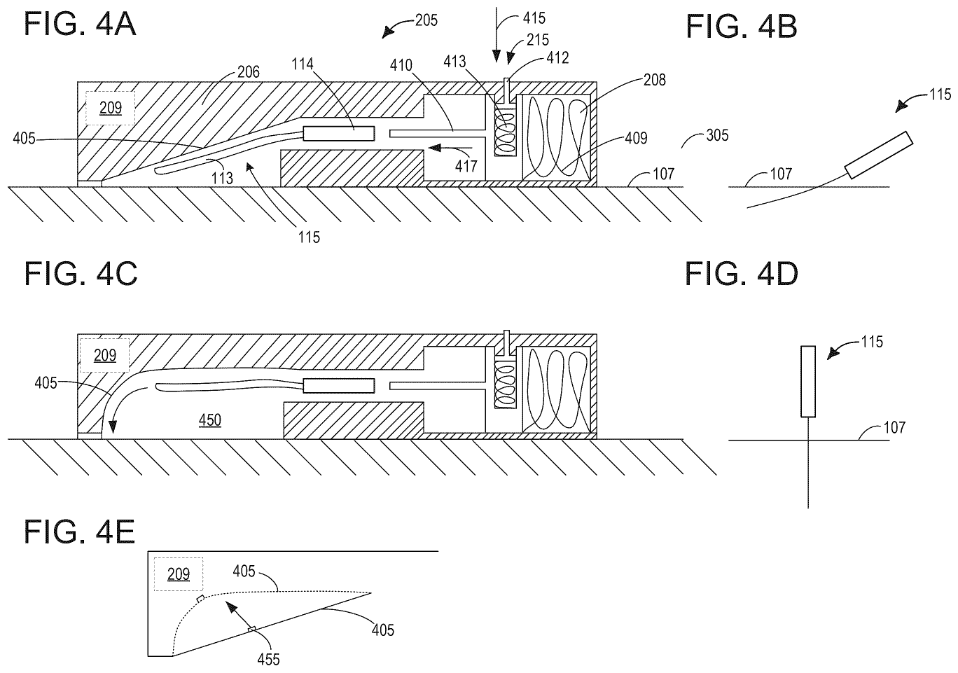

In other examples, a shape of guide structure 207 may be adjusted via insertion angle adjustment component 209. The adjustment in shape may occur without a substantial change in sensor positioning, although some small changes may occur. For example, an adjustment to the shape of guide structure 207 may cause a portion (e.g., the smaller diameter end 113) to move, without a similar movement of larger diameter end 114. Examples of guidance structures 207 that exhibit changes in shape to accommodate different flexible analyte sensor insertion angles, are discussed below with regard to FIGS. 4A-6D.

Insertion angle adjustment component 209 may be associated with insertion angle adjustment actuator 212. In some examples, insertion angle adjustment actuator 212 may comprise a knob, button, dial, switch, slider, etc. In such an example, insertion angle adjustment actuator 212 may be positioned, for example, on an exterior of a housing of insertion device 205, such that a user may readily access insertion angle adjustment actuator 212 to control flexible analyte sensor insertion angle. In such examples, insertion angle adjustment actuator 212 may be mechanically coupled to componentry (e.g., gears, rack and pinion structure, pulley and chain, etc.) that enables mechanical movement associated with insertion angle adjustment actuator 212 to be translated into adjustment of guide structure 207. In some examples, insertion angle adjustment actuator 212 may enable continuously variable guide structure 207 adjustment. As an example where insertion angle adjustment actuator 212 is a rotatable knob, a user may select a particular angle based on a number of angles inscribed on the rotatable knob, each of the number of angles corresponding to a guide structure angle in relation to the skin plane. For example, a user may select “90°” for an insertion angle that is perpendicular to the skin plane.

In another example, insertion angle adjustment actuator 212 may not enable a continuously variable adjustment of guide structure 207, but instead may enable a series of “steps”, for example steps of predetermined number of degrees (e.g., steps of 2°, 4°, 5°, 8°, 10°, etc.).

It is also within the scope of this disclosure that insertion angle adjustment actuator 212 may comprise an electromechanical actuator. For example a small motor (e.g., direct current (DC) motor) may be included as part of insertion device 205, that enables rotary motion of the motor to ultimately be translated into fine adjustment (e.g., shape or overall positioning) of guide structure 207. In examples where insertion angle adjustment actuator 212 includes a motor, it may be possible to power the motor with battery 114. In such an example, it may be desirable to include battery 114 as part of sensor base 105, although in other examples battery 114 may be included as part of control unit 109, without departing from the scope of this disclosure. In other additional or alternative examples where insertion angle adjustment actuator 212 includes a motor for adjustment of guide structure 207, insertion device 205 may include its own power supply (not shown). Such a power supply may comprise a battery, for example, or may receive power from an external power supply (e.g., electrical outlet).

In a situation where insertion angle adjustment actuator 212 comprises a small DC motor, insertion device 205 may include electronic componentry (e.g., transmitter, microprocessor, memory) that enables the motor to be controlled from an external device. In one such example, the external device may comprise electronic monitoring unit 110. In some examples, the electrical componentry may comprise componentry discussed above with regard to control unit 109. In such an example, it may be understood that guide structure 207 may be adjusted while control unit 109 is in place atop sensor base 105.

In examples, insertion device 205 may comprise an adjustable tensioning structure 210. In one example, adjustable tensioning structure 210 may comprise any substantially rounded protrusion that is perpendicularly (and in some examples horizontally) adjustable with respect to the plane comprising the external surface of skin. Adjustable tensioning structure 210 may push against skin to impart a variable amount of depression in the skin, which may be advantageous in terms of the varying angles at which flexible analyte sensor 115 is inserted. As the adjustable tensioning structure 210 functions to push against skin, adjustable tensioning structure 210 may be comprised of a material that reduces associated discomfort. Examples include but are not limited to elastic polymeric material. Specifically, to avoid sensor buckling or bending, which may lead to degraded sensor insertion attempts, it may be advantageous for the flexible analyte sensor 115 to enter skin substantially perpendicularly to the external plane of skin. In the absence of adjustable tensioning structure 210, this is would not possible except for situations where insertion angle was 90° with respect to the external plane of skin. Adjustable tensioning structure 210 may cause depression and tensioning in the skin, such that at each insertion angle, flexible analyte sensor 115 enters skin in a substantially perpendicular manner.

For example, adjustable tensioning structure 210 may be positioned surrounding exit port 207 of guide structure 206. Adjustable tensioning structure 210 may undergo movement that is coordinated with guide structure 206 adjustments. For example, when guide structure 206 is positioned at an angle of 90° with respect to skin, adjustable tensioning structure 210 may retract so that the external surface of skin is not depressed. As guide structure 206 position (or shape) is adjusted, adjustable tensioning structure 210 may correspondingly be adjusted in coordinated fashion. Thus, it may be understood that adjustable tensioning structure 210 may maximally depress skin, for example, when guide structure 206 is controlled to facilitate flexible analyte sensor insertion at 10°. Alternatively, adjustable tensioning structure 210 may minimally (e.g., not at all) depress skin, for example, when guide structure 206 is controlled to facilitate flexible analyte sensor insertion at 90°.

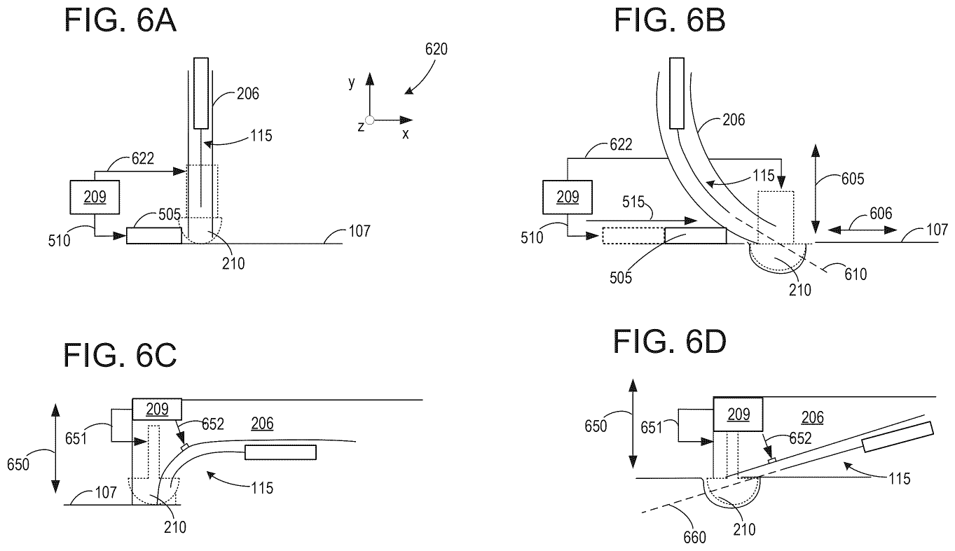

The coordinated adjustment of guide structure 206 position and/or shape along with position of adjustable tensioning structure 210 may be under control of insertion angle adjustment actuator 212. As an example, via suitable choice of gearing, actuation of actuator 212 may simultaneously control guide structure 206 position and/or shape adjustments, along with adjustable tensioning structure 210 adjustments. Examples of how guide structure 206 adjustments are coordinated with adjustable tensioning structure 210 adjustments are illustratively depicted at FIGS. 6A-6D.

It may be understood that skin tensioning may reduce an unsupported length of flexible analyte sensor 115 as it is inserted into skin. Specifically, unsupported length as discussed herein refers to a length of flexible analyte sensor that is not supported by guide structure 206 during insertion. In a case where skin is not tensioned, the insertion process of the flexible analyte sensor may result in depression of skin prior to the small diameter end 113 puncturing skin. This additional skin depression prior to skin puncture may increase unsupported sensor length, as compared to skin that is tensioned and does not depress nearly to the extent of non-tensioned skin. The increased unsupported sensor length may increase a likelihood of sensor buckling, bending, etc., which of course is undesirable. Thus, employing adjustable tensioning structure 210 may serve to reduce unsupported sensor length. Furthermore, additional/alternative skin tensioning may be supplied via adhesive patch 112. As mentioned above, the lower elasticity of adhesive patch 112 as compared to skin may reduce skin stretching upon sensor insertion. This may be particularly advantageous at insertion angles where adjustable tensioning structure 210 does not, or does not substantially, depress skin. As an example, adjustable tensioning structure 210 may not depress skin at insertion angles of 90°, making the adhesive patch an important aspect of sensor base 105 in such situations.

Turning now to FIG. 3A, depicted is a high level illustration 300 of a manner in which an insertion angle for a flexible analyte sensor of the present disclosure can be adjusted. Specifically, shown at FIG. 3A is flexible analyte sensor 115, including larger diameter end 114 and smaller diameter end 113. Flexible analyte sensor 115 is housed within guide structure 206. It may be understood that guide structure 206 may comprise a conduit (e.g., tubing, piping, hosing, etc.) through which flexible analyte sensor 115 may travel through. Guide structure 206 may be rigid, for example comprise of hard plastic or other rigid material, or may be at least somewhat flexible. However, if at least somewhat flexible, the flexible nature of guide structure 206 may be less flexible than flexible analyte sensor 115, in order to serve as a guidance structure for the sensor.

FIG. 3A depicts flexible analyte sensor 115 and guidance structure 206 at a first position 310, and a second position 312. Double-sided arrow 307 illustrates coordinated movement of guide structure 206 and analyte sensor 115. Further depicted at FIG. 3A is insertion activation device 208. Insertion activation device 208 is also shown to move between first position 310 and second position 312. Arrow 305 illustrates a direction in which insertion activation device 208 applies the high speed motive force to flexible analyte sensor 115, to drive the sensor into skin 107. As an example, insertion activation device 208 may comprise a mechanical spring-based actuator, which may store energy that, when released, causes insertion activation device to produce the high speed motive force which acts upon flexible analyte sensor 115. Other exemplary insertion activation devices are described above. While not explicitly illustrated at FIG. 3A, sensor base 105 may be included as further discussed at FIG. 3B.

Insertion angle adjustment component 209 is depicted at FIG. 3A as a dashed box encompassing flexible analyte sensor 115, guidance structure 206, and insertion activation device 208. This is to exemplarily illustrate that insertion angle adjustment component 209 may be configured to enable a repositioning of components including and/or associated with one or more of flexible analyte sensor 115, guidance structure 206, and insertion activation device 208.

Turning to FIG. 3B, depicted is another high level illustration 350, that shows an insertion device 205 that operates in an overall manner as depicted and discussed with regard to FIG. 3A. Components that are the same between FIGS. 3A and 3B are not numbered for clarity, with the exception of insertion angle adjustment component 209. In this example embodiment, sensor base 105 may include (e.g., may be provided with) flexible analyte sensor 115. More specifically, sensor base 105 may comprise a disposable component, along with flexible analyte sensor 115. The combination of sensor base 105 and flexible analyte sensor 115 may be provided in a sterile packaging, sealed until its use is desired.

In this example, sensor base 105 and associated flexible analyte sensor 115 may be placed upon skin 107 of a subject and then insertion device 205 may be coupled to sensor base 105. In another embodiment, sensor base 105 and associated flexible analyte sensor 115 may be coupled to insertion device 205 prior to both the sensor base 105 and coupled insertion device 205 being placed upon skin 107.

Flexible analyte sensor 115 may be positioned in a predetermined position with respect to sensor base 105 prior to use. To couple insertion device 205 to sensor base 105, guide structure 206 may have to be in a certain initial position in order to readily receive flexible analyte sensor 115. Upon coupling insertion device 205 to sensor base 105, flexible analyte sensor 115 may be housed within guide structure 206. If guide structure 206 is not positioned in the proper initial position, coupling of the insertion device 205 to sensor base 105 may not occur. In an example, the initial position may be similar to position 310 depicted at FIG. 3A, although other initial positioning is within the scope of this disclosure. Once proper coupling between sensor base 105 and insertion device 205 has been established (e.g., when flexible analyte sensor 115 is housed within guide structure 206), insertion device may be operated to adjust an angle at which flexible analyte sensor is inserted into skin 107.

Insertion device 205 may comprise housing 352, which as depicted at FIG. 3B fits atop sensor base 105. While not explicitly illustrated, sensor base 105 may be releasably coupled to insertion device 205, for example by a coupling mechanism (e.g., snap fitting, lock and groove, etc.). Housing 352 may comprise a general shape similar to that of a typical computer mouse, for ease of use in terms of grasping and manipulating. Circle 360 is used to depict a window into an interior portion of the insertion device 205. Sensor base 105 at FIG. 3B is depicted as having a smaller outer perimeter than another outer perimeter of insertion device 205, however it is within the scope of this disclosure that the outer perimeter of insertion device 205 and the outer perimeter of sensor base 105 are of similar dimensions (e.g., where the outer perimeter of insertion device 205 snaps or otherwise fits/releasably locks with the outer perimeter associated with sensor base 105).

Insertion device 205 includes insertion activation device actuator 215. Insertion activation device 208 is not depicted at FIG. 3B for clarity, but it may be understood that insertion activation device actuator 215 may couple to insertion activation device 208 to trigger the high speed motive force that acts upon flexible analyte sensor 115 to drive the sensor into skin 107 of the subject.

Insertion device 205 further includes insertion angle adjustment actuator 212. Similar to that depicted at FIG. 3A, insertion angle adjustment component 209 is depicted illustratively as a dashed box housed within the interior of insertion device 205. A user may manipulate insertion angle adjustment actuator 212 in order to select a desired angle of insertion. For example, insertion angle adjustment actuator 212 is depicted as including a number of different options in degrees (10°-90°). Aligning a particular degree with indicator 356 may, via actuation of insertion angle adjustment component 209, position guide structure 206 and associated flexible analyte sensor 115 at the particular desired angle of insertion.

Further depicted at FIG. 3B is release actuator 355, which may comprise a button, slider, knob, or other similar-type actuator that, when actuated, may release insertion device 205 from sensor base 105. In this way, sensor base 105 may be released from insertion device 205 such that sensor base 105 and associated inserted flexible analyte sensor 115 remain attached to skin, while insertion device 205 may be kept for future sensor application. Following release of insertion device 205 from sensor base 105, control unit 109 may be coupled to sensor base 105, as discussed above with regard to FIG. 1. In some examples, coupling of control unit 109 to sensor base 105 may include flexing/bending at least a portion of flexible analyte sensor 115 that is external to the body, in order to accommodate the coupling.

FIGS. 3A-3B depict an example scenario where selecting an angle of insertion includes movement of guide structure 206 and associated flexible analyte sensor 115 from one position to another. In other examples, it is herein recognized that angle of insertion may be adjusted by way of changing a guide structure shape.

Turning to FIG. 4A, depicted is another embodiment of an insertion device 205. In this embodiment, flexible analyte sensor 115 is supported at its larger diameter end 114 prior to insertion. Guidance structure 206 includes an adjustable section 405. Adjustable section 405 is depicted as substantially linear at FIG. 4A. A general shape of adjustable section 405 may be changed by way of insertion angle adjustment component 209, which is depicted illustratively at FIG. 4A as a dashed box, and the function of which is elaborated in greater detail below. As shown at FIG. 4A, guidance structure 206 in this embodiment comprises an at least partially open region with adjustable section 405 which guides and supports flexible analyte sensor 115 during insertion. Specifically, when the high speed motive force is applied to flexible analyte sensor 115, an outward force is exerted against a supporting wall of guidance structure 206 within the adjustable section 405. This force supports and stabilizes flexible analyte sensor 115 without a requirement that the guidance structure 206 wholly surround flexible analyte sensor 115.

Further depicted at FIG. 4A is insertion activation device 208. In this example embodiment, insertion activation device 208 comprises spring 409 and plunger 410. Also shown is insertion activation device actuator 215, which in this example comprises a pin 412 and pin-holding spring 413. Pin-holding spring 413 may bias pin 412 to a position (as depicted at FIG. 4A) that maintains compression of spring 409 of insertion activation device 208, and thereby prevents movement of plunger 410 in the direction of arrow 417. Upon actuation of pin 412 in the direction of arrow 415, energy stored in spring 409 may be released, resulting in movement of plunger 410 in the direction of arrow 417. This movement of plunger 410 may contact the larger diameter end 114 of flexible analyte sensor 115, thus providing the high speed motive force that directs the insertion of flexible analyte sensor 115 into skin 107. Although insertion activation device 208 is depicted as spring-based in this embodiment, it is within the scope of this disclosure that insertion activation device 208 may rely on a different means of producing the high speed motive force. Examples include but are not limited to an electric solenoid, a shape memory alloy spring which provides an electrically initiated driving force, an associated CO₂ cartridge, a compressed air pump, and the like.

Turning to FIG. 4B, depicted is an example illustration of flexible analyte sensor 115 inserted into skin 107, under conditions where guidance structure 206 is configured substantially similar to that depicted at FIG. 4A.

FIG. 4C depicts another example illustration of insertion device 205 which may be understood to comprise the same insertion device 205 as that depicted at FIG. 4A. The difference between FIG. 4A and FIG. 4C is simply that a shape of guidance structure 206, particularly section 405 of guidance structure 206, has been adjusted by insertion angle adjustment component 209, to adopt a more curved shape. When section 405 is configured to include such a curved shape, the angle at which flexible analyte sensor 115 is inserted into skin 107 may be quite different than circumstances where section 405 is configured as substantially linear (refer to FIG. 4A). FIG. 4D depicts an example illustration where flexible analyte sensor 115 is inserted essentially vertically (e.g., angle of 90° with respect to an external plane of skin) into skin 107. It may be understood that the overall shape of section 405 of guidance structure 206 shown at FIG. 4C may facilitate vertical insertion such as that shown at FIG. 4D.

FIGS. 4A-4D are shown to illustratively depict two substantially different scenarios of flexible analyte insertion angles, for example approximately 10-20° insertion angle as enabled by the shape of guide structure 206 as shown in FIG. 4A, and a 90° insertion angle as enabled by the shape of guide structure 206 as shown in FIG. 4C. However, the concept of a variable shape of guide structure 206 as depicted at FIG. 4A and FIG. 4C may enable any number of different insertion angles, varying for example from 10° to 90°. Said another way, insertion angle adjustment component 209 may enable continuously variable adjustment, or even step-wise adjustment, of the shape of section 405 of guide structure 206 so that any insertion angle from 10° to 90° may be possible.

A feature of the embodiment shown at FIG. 4A and FIG. 4C is that the open region 450 (refer to FIG. 4C) may allow for flexible analyte sensor 115 to be easily and completely freed from insertion device 205 following successful sensor insertion. In addition, in an embodiment, open region 450 may be large enough that additional electrical connections and/or components associated with flexible analyte sensor 115 may be accommodated before, during and/or after insertion.

In order to change shape of section 405 of guide structure 206, it may be understood that section 405 may be at least somewhat flexible and/or may have an elastomeric quality. For example, the wall that corresponds to section 405 may be comprised of a flexible and/or elastomeric material, such that shape changes are readily accommodated by way of insertion angle adjustment component 209. For example, section 405 of guide structure 206 may be comprised of a material with a Young's modulus of 1 GPa or less, or 0.1 GPa or less. Section 405 of guide structure 206 may be comprised of flexible plastic, rubber, flexible polytetrafluoroethylene, and the like.

To enable changes in shape of section 405 of guide structure 206, insertion angle adjustment component 209 may comprise a means for exerting a pulling force or forces on section 405. Turning to FIG. 4E, depicted is an example illustration showing section 405 in a first shape similar to that depicted at FIG. 4A, and in a second shape similar to that depicted at FIG. 4C. Depicted is an attachment component 455 secured to section 405 and that, while not explicitly illustrated, may be coupled/connected to or otherwise a part of insertion angle adjustment component 209. Arrow 458 depicts a general direction of pull-force that insertion angle adjustment component 209 exerts on section 405, to change the overall shape of section 405 from the first substantially linear shape to the second substantially curved shape. The pull force generated by insertion angle adjustment component 209 may be generated, for example, by appropriate gearing, pulley and chain system, etc. In some examples, there may be a plurality of associated attachment components that couple/connect or are otherwise a part of insertion angle adjustment component, to enable fine control over the overall shape of section 405. Not shown at FIG. 4A, FIG. 4B and FIG. 4E is insertion angle adjustment actuator 212, however it may be understood that such an actuator may be controllable by a user to impart the desired changes in shape to section 405 of guide structure 206, in order to adjust the angle of insertion for flexible analyte sensor 115.

FIG. 4A and FIG. 4C depict examples where sensor base 105 is not specifically shown. In some examples, it may be understood that insertion device 205 as depicted at FIG. 4A and FIG. 4C may be used to insert flexible analyte sensor 115 into skin 107 in absence of sensor base 105, and following insertion, sensor base 105 may be positioned atop the implanted flexible analyte sensor, for example by way of a hole or port in sensor base to accommodate the inserted flexible analyte sensor. Then, with the sensor base 105 appropriately positioned on the skin, control unit 109 may be coupled to sensor base 105 in similar fashion as that depicted at FIG. 1.

In another example, insertion device 205 as depicted at FIG. 4A and FIG. 4C may be (or may be a part of) sensor base 105. In such an example, insertion angle adjustment actuator 212 (not specifically illustrated at FIG. 4A and FIG. 4C) may be integral to insertion device 205 as depicted at FIG. 4A and FIG. 4C, and may be used to control insertion angle adjustment component 209 before or after placement of insertion device 205 (which is also functioning as the sensor base) upon the skin. In such an example, control unit 109 may, when placed atop insertion device 205 and pressed down, cause depression of pin 412, which may in turn trigger flexible analyte sensor insertion by releasing energy stored in spring 409, as discussed above.

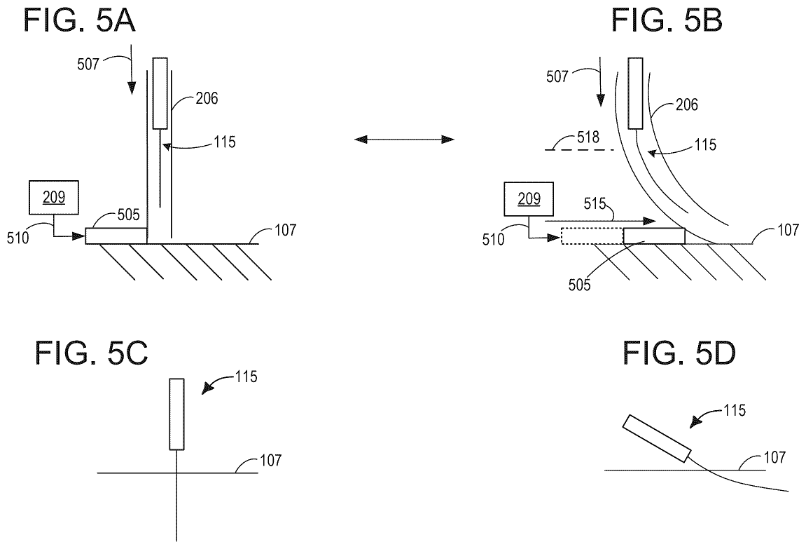

Turning now to FIGS. 5A-5D, depicted is another example embodiment of how flexible analyte insertion angle may be adjusted in line with the concepts of the present disclosure. The mechanism depicted at FIG. 5A and FIG. 5B is illustrated at a high level of generality, for illustrative purposes.

FIG. 5A depicts guide structure 206, flexible analyte sensor 115, insertion angle adjustment component 209, slider element 505, and skin 107. As illustrated at FIG. 5A, flexible analyte sensor 115 is surrounded by guide structure 206. Furthermore, guide structure 206 is positioned perpendicularly to skin 107. Not shown is insertion activation device 208, but it may be understood that such an insertion activation device may be used to impart the high speed motive force in the direction of arrow 507, to drive flexible analyte sensor 115 into skin 107.

Arrow 510 is used to indicate that insertion angle adjustment component 209 may, by way of insertion angle adjustment actuator 212 (not shown at FIG. 5A), apply a force on slider element 505 that may cause slider element 505 to move in the direction of guide structure 206. In the example depicted at FIG. 5A, insertion angle adjustment component 209 may be understood to not be applying a force on slider element 505, hence slider element 505 occupies a first, or default position as shown. While not explicitly illustrated, slider element 505 may be biased to the first position by means of a spring, elastomeric band, and the like. Accordingly, in the absence of a force applied on slider element 505 via insertion angle adjustment component 209, guide structure 206 may adopt the vertical positioning with respect to the external plane of skin 107. With guide structure 206 configured vertical to the external plane of skin 107, upon application of the high speed motive force provided via insertion activation device 208 (not shown at FIG. 5A) in the direction of arrow 507, flexible analyte sensor 115 may be vertically inserted into skin 107, as depicted at FIG. 5C.

Turning to FIG. 5B, depicted is an example illustration of how insertion angle adjustment component 209 can cause movement of slider element 505, such that an overall shape of guide structure 206 changes. The change in shape of guide structure 206 may in turn change the angle at which flexible analyte sensor 115 is inserted into skin 107.

Specifically, arrow 515 at FIG. 5B illustrates a direction of a force that is imparted upon slider element 505 by way of insertion angle adjustment component 209, causing slider element 505 to move from a first position (exemplified by the dashed box), to a second position. The first position, exemplified by the dashed box, may correspond to the same position of slider element 505 at FIG. 5A, for example. As slider element 505 moves in the direction of arrow 515, guide structure 206 changes shape, due to a flexible and/or elastomeric quality of guide structure 206. For example, guide structure 206 may comprise a flexible tubing (e.g., PTFE tubing, rubber tubing, flexible plastic tubing, and the like), capable of deforming in similar fashion to that depicted at FIG. 5B when an external force is applied. In examples, at least a portion of guide structure 206 may be prevented from substantial deformation or shape change. For example, the portion above dashed line 518 may be either be structurally secured in a manner to prevent or limit its deformation, and/or may be comprised of a different material (e.g., more rigid plastic, rubber, teflon, etc.) than the other portion of guide structure 206 below dashed line 518. With the shape of guide structure 206 changed due to the force imparted on guide structure 206 by way of slider element 505, upon application of the high speed motive force provided via insertion activation device 208 (not shown at FIG. 5B) in the direction of arrow 507, flexible analyte sensor 115 may be inserted into skin 107 at an angle, as depicted at FIG. 5D.

Similar to that discussed above with regard to FIG. 4A and FIG. 4B, in some examples the components of FIG. 5A and FIG. 5B may enable sensor insertion in absence of a sensor base (e.g., sensor base 105), and then the sensor base may be subsequently positioned over the inserted sensor. Following positioning of the sensor base 105 over the already implanted flexible analyte sensor 115, control unit 109 may be coupled to sensor base 105, as depicted at FIG. 1. In such an example, the components depicted at FIGS. 5A and 5B may comprise insertion device 205, and may be contained within a housing (not shown at FIGS. 5A-5B).

In another embodiment, it is within the scope of this disclosure that components of FIGS. 5A-5B including guide structure 206, slider element 505, insertion angle adjustment component 209 (and associated insertion angle adjustment actuator 212) may be used in conjunction with a sensor base 105, for example a disposable sensor base 105 that includes an associated flexible analyte sensor 115. Such an example may be similar to that depicted at FIG. 3B, with differences being the manner in which insertion angle adjustment component operates in conjunction with sensor base 105.

For example, a sensor base 105 may be applied to skin, and the sensor base may include an associated flexible analyte sensor 115. A separate insertion device 205 (e.g., reusable) comprising guide structure 206, slider element 505, and insertion angle adjustment component 209 (and associated insertion angle adjustment actuator 212) may be coupled to sensor base 105 in a manner that enables guide structure 206 to receive flexible analyte sensor 115. The coupling between such a sensor insertion device 205 and sensor base 105 may be substantially similar to that discussed above with regard to FIG. 3B. Upon coupling the sensor insertion device 205 to sensor base 105, insertion angle adjustment component 209 may be controlled by a user via the insertion angle adjustment actuator 212, in order to select a particular angle at which flexible analyte sensor 115 is inserted into skin. Actuation of the insertion angle adjustment actuator 212 may cause appropriate movement of slider element 515, such that the shape of guide structure 206 changes to facilitate the desired angle of sensor insertion. Once the desired insertion angle is selected, actuation of the insertion activation device 208 via the insertion activation device actuator 215 may result in the flexible analyte sensor 115 being driven into the skin at the desired insertion angle. Following insertion, the insertion device 205 may be decoupled from the sensor base 105 (e.g., by actuation of a release button similar or the same as release actuator 355 at FIG. 3B). Then, with the insertion device 205 removed, control unit 109 may be positioned atop sensor base 105 as depicted at FIG. 1. In some examples, it may be understood that depending on the angle of insertion, a portion of flexible analyte sensor 115 external to the body may be required to be flexed in a manner to accommodate placement of control unit 109 atop sensor base 105.

An advantage to the types of insertion device embodiments disclosed with regard to FIG. 4A and FIG. 4C, as well as FIGS. 5A-5B, is that because the change in shape of the guide structure dictates the angle of flexible analyte sensor insertion, positioning of the insertion activation device (e.g., insertion activation device 208) may not have to change with a corresponding change in sensor insertion angle. Similarly, a direction of the high speed motive force applied to a flexible analyte sensor may also not have to change as a function of sensor insertion angle. These advantages may be realized as compared to, for example, the embodiments shown illustratively at FIGS. 3A-3B. However, even with regard to FIGS. 3A-3B, it is herein realized that there may be possibility for an insertion activation device (e.g., insertion activation device 208) to remain essentially fixed for varying sensor insertion angles. One exemplary embodiment may include a situation where high pressure air (e.g., compressed air) is used as the high speed motive force for sensor insertion. In such an example, a source of the high pressure air may remain fixed, while a tubing that couples the high pressure air source to the guide structure (e.g., guide structure 206) may be configured to move corresponding to the positioning of the guide structure.

As discussed above with regard to FIG. 2, in some examples insertion devices 205 of the present disclosure may include adjustable tensioning structure 210. Turning now to FIGS. 6A-6D, depicted are example illustrations that show how such an adjustable tensioning structure 210 may operate in conjunction with the mechanisms of variable insertion angle discussed herein.

Specifically, FIGS. 6A-6B depict similar components as discussed above with regard to FIGS. 5A-5B, with the inclusion of tensioning structure 210. FIGS. 6C-6D depict similar components as discussed above with regard to FIG. 4A and FIG. 4C, also with the inclusion of tensioning structure 210. As depicted at FIGS. 6A-6D, tensioning structure 210 is associated with guidance structure 206, and exhibits variable displacement as a function of guidance structure shape. For example, FIG. 6A illustrates that tensioning structure 210 does not cause depression of skin when guidance structure is of a shape such that flexible analyte sensor 115 is inserted vertically. This is because for vertical sensor insertion, the sensor may perpendicularly enter the external plane of skin 107 without any additional skin depression. However, as the angle of insertion changes (e.g., becomes of a lesser angle with respect to the external plane of skin), then skin depression may be desirable so as to ensure that the flexible analyte sensor 115 always perpendicularly enters skin, no matter the insertion angle.

Thus, FIG. 6A depicts a situation where tensioning structure 210 is in a first position that does not depress skin 107 to any appreciable extent, where guide structure is of a shape to enable vertical insertion of flexible analyte sensor 115. Alternatively, FIG. 6B depicts a situation where tensioning structure 210 is in a second position that is depressing skin 107 such that although the angle of sensor insertion has changed, the flexible analyte sensor still perpendicularly penetrates skin 107 (refer to dashed line 610 with respect to an entry point of skin 107.

For tensioning structure 210 to effectively work in the manner described above with regard to FIGS. 6A-6B, tensioning structure 210 may have to move in a vertical direction, exemplified by double-sided arrows 605, as well as a horizontal direction, exemplified by double-sided arrows 606. Each of double-sided arrows 605 and 606 illustrate that tensioning structure 210 may vertically move up and down, as well as horizontally from side to side. The movement may be produced via insertion angle adjustment component 209, and the movement may be coordinated by insertion angle adjustment component 209 (exemplified by arrow 622) with additional movement of slider element 505. In this way, as the shape of guide structure changes to accommodate variable sensor insertion angles, tensioning structure 210 may variably depress skin in a coordinated fashion to ensure perpendicular skin penetration no matter the selected sensor insertion angle. The coordinated movement may be imparted by appropriate gearing selection, for example, such that slider element 505 and tensioning structure 210 exhibit coordinated movement to carry out the functions discussed.

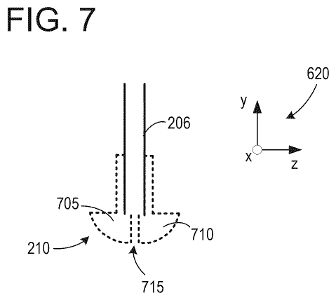

It is herein recognized that for such a tensioning structure 210 to enable variable skin depression as shown while also not interfering with (e.g., obstructing) flexible analyte sensor 115 insertion, the shape of tensioning structure 210 must carefully be considered. Reference axes 620 are shown so as to enable a discussion with regard to FIG. 7 as to how tensioning structure 210 may be constructed so that skin depression does not interfere in any way with flexible analyte sensor 115 insertion. Specifically, FIGS. 6A-6B are viewed along the z-axis for reference.

Turning to FIG. 7, depicted is an illustration of guide structure 206 and tensioning structure 210 viewed along the x-axis, using the same reference axes 620 as depicted at FIG. 6A. Tensioning structure 210 may be comprised of a first half 705 and a second half 710. First half 705 and second half 710 may structurally be mirror images of one another, and may move in coordinated fashion in a same manner (e.g., one half may not move in a direction different than the other half). In some examples, it is within the scope of this disclosure that the first half 705 and the second half 710 may be joined to one another, such that first half 705 and second half 710 are in fact comprised of a single piece. For example, a portion of tensioning structure 210 may partially surround guidance structure 206 at least when guidance structure 206 is positioned to enable vertical sensor insertion, and this portion may couple the first half 705 and second half 710 without obstructing flexible analyte sensor 115 during insertion. The advantage of tensioning structure 210 being structured as discussed is that slot 715 allows for flexible analyte sensor 115 to travel through the tensioning structure en route to skin 107, regardless of the amount to which skin 107 is depressed by tensioning structure 210.

In some examples, as illustrated at FIG. 7, slot 715 may be of a smaller width than a width (e.g., diameter) of guidance structure 206. However, in other examples the width of slot 715 may be substantially the same as the width of guidance structure 206, or of an even slightly larger width in some embodiments. Slot 715 width may be selected to both enable the smaller diameter end (e.g., smaller diameter end 113) of flexible analyte sensor 115 to readily pass through slot 715 en route to skin 107 after being routed through the exit port (e.g. exit port 207) of guidance structure 206. Slot 715 width may also be selected to be small enough so as to ensure that slot 715 does not cause any unwanted skin deformation within the vicinity of slot 715 that results in a departure from the goal of causing the external plane of skin 107 to be perpendicular to the smaller diameter end of flexible analyte sensor 115 as the smaller diameter end impacts/penetrates the skin.

Turning to FIGS. 6C-6D, as mentioned they depict similar components as discussed above with regard to FIG. 4A and FIG. 4C, with the additional inclusion of tensioning structure 210. Tensioning structure 210 as shown at FIGS. 6C-6D may be the same or substantially the same as that discussed with regard to FIGS. 6A-6B. However, because the manner in which the shape of guide structure 206 is changed with regard to FIGS. 6C-6D as compared to FIGS. 6A-6D, a difference is that tensioning structure 210 may not have to move horizontally but rather just vertically, as shown by double sided arrows 650 at FIGS. 6C-6D. However, it is within the scope of this disclosure that tensioning structure 210 may additionally move horizontally in some examples where guide structure is of the sort depicted at FIGS. 6C-6D.

Similar to that discussed above with regard to FIGS. 6A-6B, insertion angle adjustment component 209 may impart both a change in shape of guidance structure 206 (depicted by arrow 652) as well as a change in positioning of tensioning structure 210 (depicted by arrow 651). The change in guidance structure 206 shape and tensioning structure 210 positioning may occur in a coordinated fashion to enable the smaller diameter end (e.g., smaller diameter end 113) of flexible analyte sensor 115 to perpendicularly impact and penetrate skin 107 no matter the insertion angle selected by way of control over the shape of guidance structure 206 (refer to dashed line 660 at FIG. 6D). FIG. 6C depicts a situation where the flexible analyte sensor 115 is to be inserted vertically, and hence tensioning structure 210 is fully retracted so as to not cause any appreciable skin depression. Alternatively, FIG. 6D depicts a situation where flexible analyte sensor 115 is to be inserted at an angle (e.g., approximately 10-20° with respect to a non-depressed external plane of skin 107). Accordingly, FIG. 6D illustrates that tensioning structure has depressed skin 107 to an extent coordinated with the insertion angle as selected by manipulation of the shape of guidance structure 206.

While not explicitly illustrated, it may be understood that tensioning structure 210 may be included in a situation where guidance structure 206 does not change shape, but rather changes position, such as that depicted at FIGS. 3A-3B. In such an embodiment, insertion angle adjustment component 209 may similarly coordinate movement of guidance structure 206 with movement of tensioning structure 210 (e.g., vertical and in some examples horizontal movement with respect to an external plane of skin) to ensure the flexible analyte sensor 115 is perpendicularly inserted into skin, no matter the selected angle of insertion with respect to non-depressed skin.

All electrochemical analyte sensors, such as the flexible analyte sensors of the present disclosure, require electrical connections from the sensor to support circuitry on the body-worn portion (e.g., control unit 109, transmitter 130) of the device. In some examples as herein discussed, such connections have to accommodate the fact that the sensor will move during the insertion process. Discussed herein, appropriate electrical connections may be made during insertion, established prior to insertion and capable of accommodating sensor movement, or may be established subsequent to insertion. In some examples, it is within the scope of this disclosure that guide structures themselves may provide features which accommodate connection requirements. For example, open region 450 discussed above with regard to FIG. 4C may enable appropriate electrical connections to be readily established. As another example, a guide structure (e.g., a tubular structure) may integrally include contacts to facilitate electrical connections between the flexible analyte sensor and other circuitry. For example, a guide structure may include a set of contacts that electrically couple the flexible analyte sensor to other circuitry upon insertion of the sensor into skin. Said another way, the electrical connections may not be established prior to insertion, but the process of insertion may serve to establish the connections.

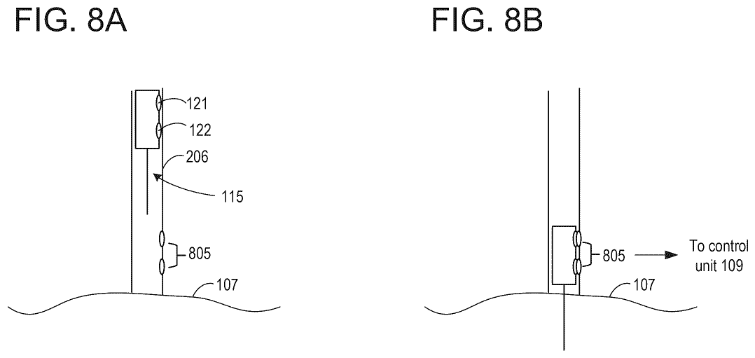

Turning to FIGS. 8A-8B, an example of such a guide structure is illustratively depicted. FIG. 8A shows guide structure 206, and flexible analyte sensor 115. Depicted at the larger diameter end 114 of flexible analyte sensor 115 is sensing electrode contact 121 and reference electrode contact 122. Guide structure itself includes corresponding guide structure contacts 805. Guide structures contacts 805 may be understood to be electrically coupled to electric circuitry associated with control unit 109 and at least transmitter 130. Prior to insertion, as shown at FIG. 8A, electrical communication between the electrical contacts associated with flexible analyte sensor 115 and guide structure contacts 805 is not yet established. Upon insertion of flexible analyte sensor 115 into skin 107, as depicted at FIG. 8B, electrical contact between the sensor contacts and the guide structure contacts becomes established.

In some examples, contacts 805 may be conductive silicon rubber molded into guide structure 206. In certain embodiments, the sensor guide structure 206 contacts 805 are insert molded metal contacts, which may be flush with the inside of the guide structure 206. In certain embodiments, the contacts 805 may comprise a flex circuit contact strip. Other similar options are within the scope of this disclosure, provided that upon sensor insertion, electrical contact is established between the electrical contacts on the sensor and the electrical contacts on the guide structure.

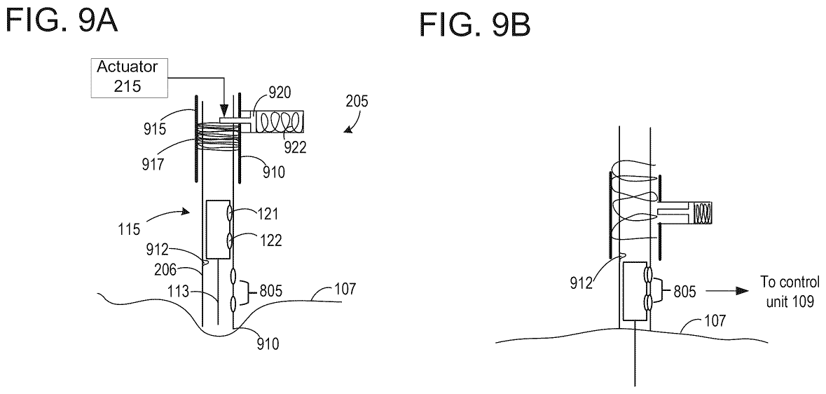

Another embodiment inserts a flexible analyte sensor of the present disclosure based on a rebound of skin due at least in part to skin elasticity. Broadly speaking, this embodiment is depicted at FIGS. 9A-9B. Turning to FIG. 9A, such an embodiment of an insertion device 205 includes guide structure 206 which houses flexible analyte sensor 115. The smaller diameter end 113 of flexible analyte sensor 115 is positioned near to or flush with an open end 910 of guide structure 206, provided that the smaller diameter end 113 does not extend past the open end 910. Flexible analyte sensor 115 may be positioned as such based on a notch 912 that prevents the sensor from extending past open end 910.

Guide structure 206 may be at least partially surrounded by a housing 915. A first compressible spring 917 may couple a portion of guide structure to housing 915. When first compressible spring 917 is compressed, as shown at FIG. 9A, housing 915 and guide structure 206 may move relative to one another. At a certain point of compression of first compressible spring 917, pin 920, biased via second compressible spring 922, may lock guide structure 206 to housing 915 to prevent further relative movement. With pin 920 in the locked position, as indicated at FIG. 9A, insertion device 205 may be pressed against skin 107, thereby indenting/depressing skin 107 in the vicinity of guide structure 206. Because of notch 912, the smaller diameter end 113 of flexible analyte sensor 115 does not contact skin 107 even when the skin is depressed. In some examples, as shown at FIG. 9A, guide structure 206 may include associated contacts 805, however in other examples guide structure 206 may not include such contacts, without departing from the scope of this disclosure. In a case where contacts 805 are included, sensing electrode contact 121 and reference electrode contact 122 may not be in contact with guide structure contacts 805, as shown at FIG. 9A.

A user of insertion device 205 in the embodiment of FIGS. 9A-9B, may actuate the device via insertion activation device actuator 215. The actuation may cause pin 920 to compress spring 922, in turn unlocking guide structure 206 from housing 915. This may action may result in rapid decompression of spring 917, which may cause housing 915 and guide structure 206 to rapidly move relative to one another at high speed, the speed dictated by properties of spring 917. The high speed movement may be sufficient to overcome a holding force of notch 912, such that the relative movement of housing 915 and guide structure 206 following actuation results in notch 912 being atop flexible analyte sensor 115 as shown at FIG. 9B, as compared to being below, as shown at FIG. 9A. The rapid movement of guide structure 206 upon actuation of the insertion activation device 205 may cause skin 107 to rapidly de-compress, returning to its usual resting state and in the process, resulting in the smaller diameter end 113 of flexible analyte sensor 115 being inserted into skin 107 as shown at FIG. 9B. In a case where guide structure 206 includes contacts 805, insertion of flexible analyte sensor 115 into skin 107 may cause alignment of sensor contacts (e.g., sensing electrode contact 121 and reference electrode contact 122) with guide structure contacts 805. It may be understood that guide structure 206 may include contacts in a case where insertion activation device 205 as depicted at FIGS. 9A-9B is not removed following sensor insertion. In a case where guide structure 206 does not include contacts 805, guide structure 206 may be removed following sensor insertion.

It is within the scope of this disclosure that the embodiment of FIGS. 9A-9B may be used in some examples with an insertion angle adjustment component 209 and/or tensioning structure 210. In other words, although the actuation mechanism described at FIGS. 9A-9B is different than other actuation mechanisms of flexible analyte sensor insertion as discussed above, similar principles may apply to changing an angle of insertion, as would be understood by those of ordinary skill in the art.

In some examples, insertion activation device as disclosed with regard to FIGS. 9A-9B may comprise or be associated with (e.g., be a part of) sensor base 105. For example, similar to that discussed above with regard to FIG. 4A and FIG. 4C, it is within the scope of this disclosure that actuation of pin 920 may occur upon coupling control unit 109 to sensor base 105, where the embodiment of FIGS. 9A-9B comprises or is associated with such a sensor base.

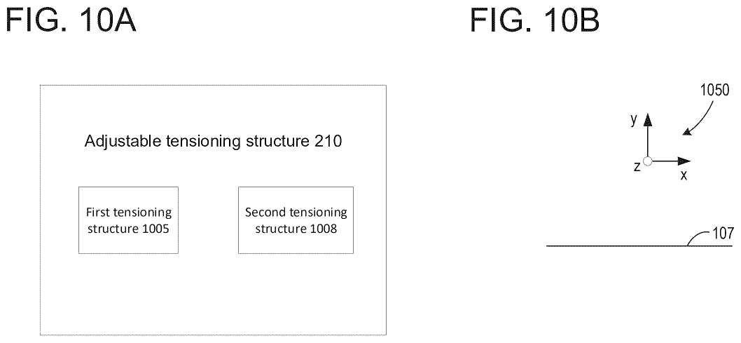

It is herein recognized that in some examples that include a skin tensioning structure such as that discussed above with regard to FIGS. 6A-6D, it may be desirable to include another adjustable skin tensioning structure. Turning to FIG. 10A, depicted is an example where adjustable skin tensioning structure 210 comprises a first adjustable tensioning structure 1005, and a second adjustable skin tensioning structure 1008. First adjustable skin tensioning structure 1005 may be understood to comprise a similar or same skin tensioning structure as that discussed above and illustratively depicted with regard to FIGS. 6A-6D. Turning to FIG. 10B, skin 107 is depicted along with reference axes 1050. With respect to reference axes 1050, first adjustable skin tensioning structure 1005 may enable depression or indentation of skin along the y axis. Second adjustable skin tensioning structure 1008 may enable stretching of skin along one or more of the x-axis and z-axis, or any manner of stretching along the x-z plane.

It was above-discussed that an adhesive patch (e.g., adhesive patch 112 at FIG. 1), for example an adhesive patch associated with a sensor base (e.g., sensor base 105 at FIG. 1), may enable a tightening of the skin which may be desirable at particular angles of sensor insertion, for example vertical angle insertion (90° with respect to an external plane of the skin). However, in a case where a skin tensioning structure such as that depicted at FIGS. 6A-6D is included as part of insertion devices (e.g., insertion device 205) of the present disclosure, as the tensioning structure further depresses skin it may not be desirable to also attempt to stretch skin along the external plane to a same extent independent of the extent of skin depression.

Specifically, a fixed stretching amount along an external plane of skin may act to counter any effect of depressing the skin. Thus, it is herein recognized that when a tensioning structure that acts by depressing skin (e.g., first tensioning structure 1005) is included in an insertion device of the present disclosure, inclusion of another tensioning structure (e.g., second tensioning structure 1008) that is capable of variably stretching skin along the external plane (e.g., x-z plane at FIG. 10B), may be advantageous. Specifically, first tensioning structure 1005 and second tensioning structure 1008 may exhibit coordinated movement with respect to one another, and with respect to the variable insertion angle (e.g., with respect to the shape and/or position of guidance structure 206). The movement may be coordinated by the insertion angle adjustment component 209, similar to that discussed above, for example with regard to FIGS. 6A-6D. Specifically, as first tensioning structure 1005 further depresses skin, second tensioning structure 1008 may exhibit a lesser stretching force on the external plane of the skin. Alternatively, as first tensioning structure 1005 depresses skin to a lesser extent, second tensioning structure 1008 may exhibit a greater stretching force on the external plane of the skin.

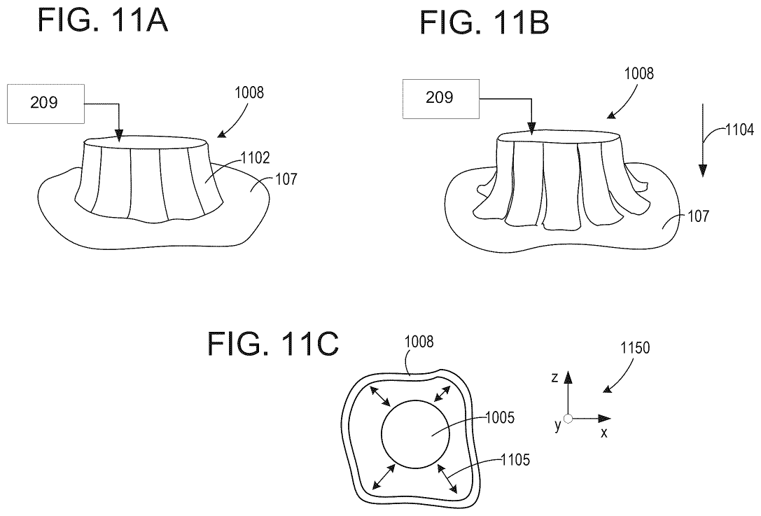

Turning to FIGS. 11A-11B, depicted is one example embodiment of such a second tensioning structure 1008. In this example, second tensioning structure 1008 includes a plurality of elastomeric units 1102. In a first position as shown at FIG. 11A, the plurality of elastomeric units 1102 exhibit a lesser flexing, and in a second position as shown at FIG. 11B, the plurality of elastomeric units 1102 exhibit a greater amount of flexing. The greater amount of flexing may cause a greater amount of skin stretching along the external plane of the surface. Specifically, a force acting on second tensioning structure 1008 in the direction shown by arrow 1104 may impart the greater stretching of skin along the external plane, whereas reduction of the force acting on second tensioning structure 1008 may cause second tensioning structure 1008 to adopt the first position as shown at FIG. 11A. Other similar types of embodiments as that discussed with regard to FIGS. 11A-11B are within the scope of this disclosure, provided they are capable of causing similar skin tensioning and release along the external skin plane in a reversible manner.

In an embodiment, it may be desirable for first skin tensioning structure 1005 to be surrounded or at least partially surrounded by second skin tensioning structure 1008. For example, turning to FIG. 11C, depicted is an example illustrating a top-down view (e.g., along the y-axis, refer to axes 1150) of a cross-section of first skin tensioning structure 1005 and second skin tensioning structure 1008. As discussed above, skin tensioning structures of the type corresponding to first skin tensioning structure 1005 may be associated with an exit port (e.g., exit port 207) of a guide structure (e.g., guide structure 206) where a flexible analyte sensor (e.g., flexible analyte sensor 115) is inserted into skin. Thus, having second skin tensioning structure 1008 at least partially surrounding first skin tensioning structure 1005 may enable second skin tensioning structure to impart a variable amount of skin tensioning along the skin external plane (e.g., x-z plane with regard to FIG. 11C) in the region corresponding to where the flexible analyte sensor is inserted. This is depicted illustratively by double-sided arrows 1105 at FIG. 11C, showing that relative movement of second skin tensioning structure 1008 can cause skin tensioning and release in a reversible manner within the vicinity of first skin tensioning structure 1005, and correspondingly, in the vicinity of where the flexible analyte sensor is inserted into skin.

In this way, flexible analyte sensors, for example flexible analyte sensors comprising a component of continuous analyte monitoring systems (e.g., continuous glucose monitoring systems), may be readily inserted into skin at a wide variety of different angles. This may improve customer satisfaction, as different body sites may be used depending on user preference and/or to avoid repeated sensor insertion in same body locations. Further, the insertion devices of the present disclosure may enable variable analyte sensor insertion depending on one or more of age, body mass index (BMI), body location, etc.

While certain embodiments have been described above, other embodiments are possible. For example, the guidance structure 206 can be implemented as a dimple in the sensor base 105 where the flexible analyte sensor 115 enters the skin. As such, the guidance structure 206 can be a part of the sensor base 105.

In some embodiments, a structure similar to the adjustable tensioning structure is used to insert the flexible analyte sensor 115 into the skin. This adjustable structure can be used to stretch the skin and to support the flexible analyte sensor 115. In other words, the flexible analyte sensor 115 can be located within the adjustable structure that is pressed into the skin. In such embodiments, the flexible analyte sensor 115 is locked into position and the support of the adjustable structure is removed, allowing the skin to push back the adjustable structure and leave the flexible analyte sensor 115 in the skin.

In some embodiments, the sensor 115 may be supported, but as the size (e.g., length) of the sensor 115 is decreased, the support can be reduced to the point where no support is required. For example, the wire tip of the sensor 115 can be sharpened so that insertion of the sensor 115 does not require significant force or support to penetrate the skin. In some embodiments, the length of the sensor 115 is 5 mm, which may also help prevent or reduce buckling.

The material characteristics of the sensor filament can be adjustable to accommodate the mode of sensor insertion. There are several options related to modifying the sensor filament strength and bend characteristics that can be applied to match the sensor to the desired insertion mode. This may include changing the annealing process to match the specific filament characteristics needed. Possible sensor types include flat sensors, polymer base materials, sensors with multiple electrodes, 3-electrode potentiostats, redundant electrodes, multiple sensing devices on a single sensor, etc. In some embodiments, conductive polymers, screened or inkjet application of coatings, electroplated conductive polymers, electrodeposition of coatings, and/or nanomaterials may be used to fabricate the sensor 115. For vertical insertion, the insertion device 205 may have multiple sensors and a relatively small footprint on the skin.

A number of embodiments have been described. Nevertheless, it will be understood that various modifications can be made without departing from the spirit and scope of the present disclosure. Accordingly, other embodiments are within the scope of the following claims.

What is claimed is:

1. An insertion device for delivering an analyte sensor into skin, comprising:
a guidance structure that at least partially supports the analyte sensor during insertion of the analyte sensor into the skin; and
an insertion angle adjustment component configured to adjust an angle at which the analyte sensor is driven into the skin.

2. The insertion device of claim 1, wherein the analyte sensor further comprises a flexible analyte sensor having a buckling force of less than 0.25 Newtons.

3. The insertion device of claim 1, wherein the insertion angle adjustment component adjusts the angle at which the analyte sensor is driven into the skin by changing a shape of the guidance structure.

4. The insertion device of claim 1, wherein the insertion angle adjustment component adjusts the angle at which the analyte sensor is driven into the skin by changing a position of the guidance structure without altering a shape of the guidance structure.

5. The insertion device of claim 1, further comprising an insertion activation device configured to provide a high speed motive force to the analyte sensor to drive the analyte sensor into the skin.

6. The insertion device of claim 1, further comprising an adjustable skin tensioning structure configured to tension the skin prior to delivering the analyte sensor into the skin.

7. The insertion device of claim 6, wherein the insertion angle adjustment component coordinates adjustment of the adjustable skin tensioning structure as a function of the angle at which the analyte sensor is driven into the skin.

8. The insertion device of claim 7, wherein tensioning the skin via the adjustable skin tensioning structure includes depressing the skin to a variable extent, where the skin is depressed to a greater extent as the angle at which the analyte sensor is driven into the skin is reduced; and
wherein the skin is depressed to a lesser extent as the angle at which the analyte sensor is driven into the skin is increased.

9. The insertion device of claim 1, wherein the angle at which the analyte sensor is driven into the skin is selectable from 10° to 90° with respect to an external plane of the skin.

10. An insertion device for delivering a flexible analyte sensor into skin, comprising:
an insertion angle adjustment component configured to adjust an angle at which the flexible analyte sensor is delivered into the skin;
a guidance structure operably coupled to the insertion angle adjustment component; and
wherein the angle at which the flexible analyte sensor is delivered into the skin is adjusted via the insertion angle adjustment component by changing a shape of the guidance structure.

11. The insertion device of claim 10, wherein changing the shape of the guidance structure adjusts the angle at which the flexible analyte sensor is delivered into the skin from 10° to 90° with respect to an external plane of the skin in a continuously variable manner.

12. The insertion device of claim 10, wherein changing the shape of the guidance structure adjusts the angle at which the flexible analyte sensor is delivered into the skin from 10° to 90° with respect to an external plane of the skin in a non-continuously variable step-wise manner.

13. The insertion device of claim 10, wherein the guidance structure is configured to receive a high speed motive force from an insertion activation device, the high speed motive force comprising a force that causes movement of the flexible analyte sensor that ultimately delivers the flexible analyte sensor into the skin.

14. The insertion device of claim 13, wherein the insertion activation device remains fixed in terms of position regardless of the angle at which the flexible analyte sensor is delivered into the skin.

15. The insertion device of claim 13, wherein a direction at which the high speed motive force is produced via the insertion activation device relative to an external plane of the skin does not change, regardless of the angle at which the flexible analyte sensor is delivered into the skin.

16. The insertion device of claim 13, wherein the high speed motive force is provided mechanically, pneumatically, or hydraulically.

17. The insertion device of claim 10, further comprising an adjustable skin tensioning structure configured to depress the skin to a variable extent as a function of the angle at which the flexible analyte sensor is delivered into the skin; and
wherein the insertion angle adjustment component simultaneously coordinates movement of the skin tensioning structure with changing of the shape of the guidance structure.

18. The insertion device of claim 17, wherein the adjustable skin tensioning structure depresses the skin to the variable extent as the function of the angle at which the flexible analyte sensor is delivered into the skin to ensure the flexible analyte sensor perpendicularly penetrates the skin, regardless of the angle at which the flexible analyte sensor is delivered into the skin.

19. The insertion device of claim 17, wherein the guidance structure includes an exit port through which the flexible analyte sensor travels to be delivered into the skin, where the skin tensioning structure is associated with the exit port of the guidance structure; and
wherein the skin tensioning structure includes a dedicated slot through which the flexible analyte sensor travels through during delivery into the skin.

20. The insertion device of claim 10, wherein the flexible analyte sensor is delivered to the skin along a linear path or a curved path depending on the shape of the guidance structure.

21. The insertion device of claim 10, wherein the guidance structure includes one or more electrical contacts that electrically couple to a portion of the flexible analyte sensor that remains external to the skin; and
wherein electrically coupling the one or more electrical contacts included as part of the guidance structure to the portion of the flexible analyte sensor that remains external to the skin occurs as a result of delivering the flexible analyte sensor into the skin.

22. An insertion device for delivering a flexible analyte sensor into skin, comprising:
a guidance structure configured to at least partially surround the flexible analyte sensor;
a housing that at least partially surrounds a portion of the guidance structure;
a first spring that couples the housing to the guidance structure;
a pin configured to lock the housing to the guidance structure to prevent relative movement of the guidance structure and the housing;
a second spring associated with the pin; and
a notch that maintains the flexible analyte sensor at a first position relative to the guidance structure under conditions where the housing is locked via the pin to the guidance structure.

23. The insertion device of claim 22, wherein compression of the second spring unlocks the housing and the guidance structure, so the guidance structure is able to move with respect to the housing at a speed that is a function of one or more parameters of the first spring.

24. The insertion device of claim 23, wherein movement of the guidance structure with respect to the housing overcomes a holding force of the notch, that in turn positions the flexible analyte sensor at a second position with respect to the guidance structure.

25. The insertion device of claim 24, wherein the guidance structure includes an exit port, wherein no part of the flexible analyte sensor crosses a plane of the exit port when the flexible analyte sensor is in the first position; and
wherein at least a portion of the flexible analyte sensor crosses the plane of the exit port to extend past the guidance structure when the flexible analyte sensor is in the second position.
